(19) United States
(12) Patent Application Publication
(10) Pub. No.: US 2003/0166054 A1
(43) Pub. Date: Sep. 4, 2003

(54) Enhanced in vitro protein synthesis

(76) Inventors: Kangseok Lee, Palo Alto, CA (US); Stanley N. Cohen, Portola Valley, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/013,774

(22) Filed: Dec. 10, 2001

Related U.S. Application Data
(60) Provisional application No. 60/254,356, filed on Dec. 8, 2000.

(51) Int. Cl. C12P 21/02; C12P 19/34; C12N 15/87

(52) U.S. Cl. 435/69.1; 435/91.2; 435/455

(57) ABSTRACT

Poly G tails prolong mRNA chemical and functional half life in E. coli cell extracts and dramatically increased RNA-dependent protein synthesis in vitro. The effect of polyguanylation on mRNA functional half life, as measured by the ability of CAT transcripts to produce biochemically-active protein in vitro, was four- to six-fold greater than the effect on chemical half life. Addition of a few nucleotides 5′ to the bacteriophage T7 promoter markedly enhanced transcription of linear PCR-generated DNA molecules by T7 RNA polymerase. Collectively a novel approach is provided for efficient in vitro protein synthesis that bypasses the need for cloned DNA templates to obtain the products of translational open reading frames.

### BACKGROUND OF THE INVENTION

The enhancement and control of gene expression and protein biosynthesis is of great interest. The development of recombinant DNA techniques has allowed the characterization and synthesis of highly purified coding sequences, which in turn can be used to produce highly purified proteins by biological processes. The biological synthesis may be performed within the environment of a cell, or using cellular extracts and coding sequences to synthesize proteins in vitro. Because it is essentially free from cellular regulation of gene expression, in vitro protein synthesis has advantages in the production of cytotoxic, unstable, or insoluble proteins. The over-production of protein beyond a predetermined concentration can be difficult to obtain in vivo, because the expression levels are regulated by the concentration of product.

While methods for altering the transcription levels of mRNA have been widely studied, there are other means for increasing the translational effectiveness of the mRNA, for example by increasing stability through altering genetic sequences and reaction conditions.

Some of the factors involved in controlling prokaryotic mRNA stability include the presence of nucleases, secondary structures, translation influences, and transcription effects

The presence and activity of nucleases is of particular interest. Prokaryotic mRNA processing controls the decay of functional RNA in order to affect the expression of a gene.

Five mRNA processing activities are associated with the inactivation and degradation of bulk mRNA in most prokaryotes. Two are associated with exonucleases which exhibit a 3′ to 5′ processive cleavage activity for degradation, while the others are associated with decay-initiating mRNA cleavage and processing by endonucleases.

The two exonucleases responsible for bulk mRNA degradation into mononucleotides are RNase II and polynucleotide phosphorylase (PNPase). Both enzymes degrade the mRNA in a processive 3′ to 5′ direction. The functions of the exonucleases appear to be redundant, in that a cell containing a mutation in either can survive, but mutations in both enzymes are lethal.

Four endonucleolytic activities have been identified in E. coli: RNase III, RNase E RNase G and RNase K. RNase III cleaves mRNA at a weak consensus sequence often within hairpins that contain unpaired internal regions. The role of RNase III in mRNA stability can be characterized as processing, as it typically cleaves hairpins contained within untranslated regions and does not directly inactivate the mRNA, although the removal of the secondary structures by RNase III can open the mRNA to rapid decay. A single enzyme is responsible for the two endonucleolytic activities identified as RNase E and RNase K. The transition between these two activities is thought to be controlled by proteolytic processing of the primary RNase E nuclease. The nuclease cleaves A+U segments, and has been reported to scan the mRNA transcript in a 5′ to 3′ direction and cleaves within AU-rich segments.

The presence of hairpin structures also affects mRNA stability. 3′-hairpins function as transcription terminators, causing the RNA polymerase to release the mRNA at the end of the gene. These hairpins also protect the mRNA from degradation by the exonucleases RNase II and PNPase. The 3′-hairpin provides a barrier to the exonucleases, preventing them from degrading the coding sequence of the mRNA. 5′-hairpins protect the mRNA from inactivation by RNase E.

The mRNA nucleotide sequence can alter the stability of the transcript by influencing the cleavage activities of the nucleases. For example, stability may be controlled by the absence or presence of recognition sequences of the ribonucleases. Although no specific recognition sequence has been determined for any of the mRNA endonucleases, general characteristics have been reported. RNase E cleavages have been mapped and shown to occur within AU-rich regions of single-stranded mRNA. Point mutations within these cleavage regions can dramatically influence the enzyme activity at a site, both to increase as well as decrease the frequency of mRNA cleavage.

Another sequence that affects stability is polyadenylation of RNA at the 3′ end. The addition of poly(A) tails to bacterial RNA leads to accelerated RNA degradation by PNPase and possibly other 3′ to 5′ exonucleases. However, the only biological consequence of slowing RNA decay by impeding polyadenylation demonstrated thus far is altered control of plasmid DNA replication. While the failure to add poly(A) tails can also stabilize a variety of mRNA species in E. coli, enhanced synthesis of proteins encoded by these RNAs has not been reported in pcnB mutant bacteria, raising the possibility that poly(A) tails may, while accelerating the decay of mRNAs, also lead to a compensatory increase in mRNA translation.

The design of systems that take advantage of mRNA stabilization techniques will find application in areas ranging from the improvement of existing biocatalytic processes to the development of recombinant gene technologies. mRNA stability has a significant influence on protein synthesis under conditions where gene expression is not limited by ribosome availability, e.g. during in vitro synthetic reactions. The use of mRNA stability control to engineer gene expression will also prove valuable in the design of engineered metabolic pathways and in the expression of genes present at low copy number in the cell.

Literature

A review of prokaryotic mRNA stability may be found in Carrier and Keasling (1997) Biotech. Prog. 13:699-708.

Addition of poly-A tails has been shown to occur in prokaryotes (Cohen (1995) Cell 80:829-832. Huang et al. (1998) Nature 391:99-102 describe the ability of RNAse E to shorten poly-A tails in mRNA. Hajnsdorf et al. (1995) P.N.A.S. 92:3973-3977 describe other effects of poly-A tails on prokaryotic mRNA stability.

### SUMMARY OF THE INVENTION

Compositions and methods are provided for the enhanced synthesis of polypeptides in vitro. The methods provide for stabilized mRNA; and enhanced transcription from phage specific promoters. The enhanced polypeptide synthesis is useful for in vitro polypeptide production from cloned and uncloned sequences. High levels of polypeptide may be synthesized from a targeted sequence by PCR amplification and subsequent transcription and translation, which methods find use in identification of encoded polypeptides, for determining protein interactions, and the like.

A stabilizing sequence, comprising a homopolymer of poly (G), poly (U), or poly (C) is inserted at the 3′ region of an mRNA, which insertion greatly increases the stability of the mRNA. In a preferred embodiment, the stabilizing sequence is inserted at the 3′ terminus of the RNA. The presence of the stabilizing sequence increases the functional half life of the RNA, and can significantly enhance the production of the encoded protein. Alternatively, the RNA stabilizing sequences are provided to increase the stability of non-coding RNA molecules, e.g. ribozymes, anti-sense RNAs, etc.

In one embodiment of the invention, the stabilized RNA is mRNA, and the invention provides for an increased yield in protein encoded by the mRNA. The protein is preferably produced in vitro. The use of in vitro translation; and coupled in vitro transcription/translation reactions is of particular interest.

In another embodiment of the invention, a method is provided for efficient transcription of PCR-amplified DNA templates in vitro, using phage RNA polymerase (RNAP). Such transcription may be combined with the insertion of stabilizing sequences for improved in vitro transcription and translation reactions. Efficient transcription occurred on a template containing base pairs 5′ to the promoter. This effect is independent of the nucleotide sequence of the 5′ base-pairs, and is due specifically to transcription by phage RNAP. The promoter and 5′ transcription enhancing sequence may be provided as a primer for amplification reactions, which primer may be universal, or specific for a targeted sequence.

In another embodiment of the invention, a method is provided for the insertion of polynucleotides onto the 3′ end of mRNA made by in vitro transcription from phage specific promoters.

### BRIEF DESCRIPTION OF THE DRAWINGS

FIG. 1. Effects of different 3′ homopolymer tails on mRNA on translation. In vitro translation was carried out at 30° C. for 30 minutes for CAT protein production and 10 minutes for LacZα protein production using in vitro synthesized, gel purified mRNAs (120 nM). Optimal incubation time was determined for maximum protein yield by measuring protein production at five minute intervals. 10 minute incubation time was used for LacZα production because this peptide was degraded more rapidly in reaction mixtures. Functional CAT protein assayed (A) as indicated in EXPERIMENTAL PROCEDURES, and LacZα protein production assayed by Western blot (B). % CAT activity and % LacZα signal were obtained by setting values (CAT activity and Western blot signal) from reactions containing mRNAs lacking tails as 100%. The LacZα fragment of β-galactosidase was epitope-tagged with a T7·tag peptide sequence its N terminus and anti T7·tag antibody conjugated with HRP was used in Western blots to detect the fusion protein. The lengths of polynucleotide tails, which were incorporated into T7-generated mRNA molecules during primary transcription, were confirmed by electrophoresis on 6% acrylamide gels by comparing the size of transcripts due to addition of tails.

FIG. 2. Effects of different 3′ tails of mRNA on mRNA decay. In vitro translation reaction was carried out using gel purified, uniformly labeled CAT (A) or lacZα (B) mRNA with ³²P-UTP (120 nM) to measure mRNA decay in the reaction. Samples were removed at times indicated and mRNA was purified by phenol extraction and ethanol precipitation and analyzed in 6% PAGE containing 8 M urea. The amount of full length mRNA left in each lane was measured using a phosphoimager and plotted (C and D). E, Functional CAT protein production in vitro was analyzed by measuring CAT activity in identical volumes removed from reaction mixtures at five-minute intervals and by plotting the incremental CAT activity of each sequentially removed volume. F, CAT protein produced in the reactions was visualized in Western blot analysis. Samples were taken after 30 minute incubation and detected using anti T7 antibody conjugated with HRP.

FIG. 3. Effect of poly(A) tail on the rate of 30S initiation complex formation. CAT mRNA containing 40 A residues or lacking any 3′ additions were used in primer-extension inhibition (toeprinting) assays with varying concentrations of small ribosomal subunit with (30S) or without S1 protein (30SΔS1). The toe-printing signal was quantitated as percent toeprinting band relative to the sum of the mature product and toeprinting bands using a phosphoimager and plotted (B). The portions of the mature products, the toeprinting signal, and the primer bands are indicated. Left four lanes in panel (A) are sequencing ladders, the same CAT mRNA and primer were used in toeprinting assays and sequencing. The location of the Shine-Dalgarno (SD) sequence and translation start codon are shown in the CAT sequence depiction.

FIG. 4. Parameters affecting efficiency of T7 promoters. A, Effects of base-pairs upstream of T7 promoter on T7 RNA polymerase directed transcription. One μg of DNA was used in 20 μl of coupled in vitro transcription/translation reaction. The reaction was carried out either in the presence (▪) or in the absence (□) of externally added T7 RNA polymerase (1U/μl) and rifampicin (500 ng/μl). B, Map of plasmid pET3a-CAT showing locations of relevant restriction enzyme cleavage sites, the T7 promoter, the CAT gene, and the PCR product generated using primers indicated in EXPERIMENTAL PROCEDURES. C, Determination of a minimum number of extra base-pairs upstream of T7 promoter required for optimal transcription by T7 RNAP. Extra base-pairs were added 5′ to T7 promoter in pET3a using PCR primers (see EXPERIMENTAL PROCEDURES) and CAT activity was measured in coupled transcription/translation reactions. D, Analysis of transcription and mRNA decay in coupled transcription/translation reactions. Samples were removed at times indicated and [³²P]-UTP incorporation into transcripts was analyzed by 6% PAGE in gels containing 8 M urea. Rifampicin was added (10 ng/μl) was added to reactions containing T7 RNAP (1U/μl), DNA concentration used was 50 ng/μl. In the last lane, in vitro transcribed CAT mRNA from PCR DNA (PT7) was loaded as a size marker (+).

FIG. 5. Effect of 3′ additions and inserted sequences on translation of CAT mRNA. A, Effect of number of G residues at the 3′ terminus of mRNA on translation. PCR-generated DNAs containing 5 base-pairs upstream of the T7 promoter and different tails 3′ to the CAT protein coding region were used in a coupled transcription/translation reaction, and the amount of translation product was measured using CAT assay. B, Effects of different 3′ termini on CAT mRNA translation. 15 G residues were internally incorporated at the 3′ terminus, followed by 5 A residues (15G5A) or five random residues (15G5N). Similar reactions described above were carried out except PCR products with different tails were used in this experiments. C, Quantitation of CAT proteins synthesized in a coupled transcription/translation reaction. Epitope-tagged CAT protein was affinity-purified from the E. coli cell extracts (BL21 (DE3)) harboring pET3a-CAT using anti T7·tag antibody. The indicated amounts of purified CAT protein were loaded onto a 10% tricine-SDS-polyacrylamide gel along with one μl of samples taken from in vitro reaction shown in FIG. 5B (C15 and G15). CAT protein was detected in Western blot using anti T7·tag antibody.

FIG. 6. Stabilization of CAT mRNA by poly(G) tail. A, Analysis of steady state levels of mRNA in coupled transcription/translation reaction. [³²P]-UTP was added to reactions, samples removed at the times indicated and the percent of undegraded CAT transcripts were quantitated using a phosphoimager, was plotted relative to CAT mRNA containing a tail of 15Gs present 10 minutes of incubation, which was set at 100% (C). B, Reaction conditions were described in FIG. 2 except a coupled transcription/translation reaction mixture was used in theses experiments. Percent RNA left in reactions were plotted (D).

FIG. 7. Effects of poly(G) tails on translation of luciferase, GPSI and TP mRNA. A, Effects of different 3′ termini on translation of luciferase mRNA. Reaction mixtures were similar to those described in FIG. 5B except that PCR products containing luciferase protein sequence. The amounts of luciferase protein produced in reactions was measured and compared by setting the amount of luciferase activity encoded by transcripts containing a 15G tail as 100%. B, Effects of poly(G) tails on translation of guanosine pentaphosphate synthetase I (GPSI) and telomere binding protein (TP). GPSI and TP coding regions were amplified either from plasmid (GPSI, pJSE371) or directly from Streptomyces rochei chromosome (TP) and the translation product was detected in Western blots using anti T7·tag (GPSI) or anti Flag·tag antibody (TP). The same membrane was reprobed with antibody to E. coli PNPase antibody to produce a control for possible variations in loading.

### DETAILED DESCRIPTION OF THE EMBODIMENTS

The in vitro synthesis of polypeptides is enhanced by stabilization of mRNA by the addition of homopolymer tails consisting of poly (G), poly (U), or poly (G); and by efficient transcription from phage specific promoters, particularly with PCR amplified sequences. The stabilized RNA can be transcribed from DNA templates, which may be uncloned segments of genomic DNA, or plasmids, phage, phagemids, virus or other high copy number sources. The RNA may be transcribed from PCR amplified DNA templates generated from these DNAs or from reverse transcribed mRNA, which may be a single species, or a complex pool of sequences, depending on the selection of PCR primers. In a preferred embodiment, PCR primers are selected such that one primer provides for an improved promoter, e.g. a promoter optimized for T7 RNA polymerase; and the other primer provides for insertion of stabilizing sequences in the mRNA.

RNA is stabilized against nucleases by insertion of a stabilizing sequence comprising at least five nucleotides of poly (G) at the 3′ terminus. The presence of the stabilizing sequence increases the functional half life of the RNA.

The phage promoter is optimized by addition of at least five base pairs 5′ to the promoter. This effect is independent of the nucleotide sequence of the 5′ base-pairs. The promoter and 5′ transcription enhancing sequence may be provided as a primer for amplification reactions, which primer may be universal, or specific for a targeted sequence.

The present invention provides novel compositions and methods as set forth within this specification. In general, all technical and scientific terms used herein have the same meaning as commonly understood to one of ordinary skill in the art to which this invention belongs, unless clearly indicated otherwise. For clarification, listed below are definitions for certain terms used herein to describe the present invention. These definitions apply to the terms as they are used throughout this specification, unless otherwise clearly indicated.

### DEFINITION OF TERMS

As used herein the singular forms “a”, “and”, and “the” include plural referents unless the context clearly dictates otherwise. For example, “a compound” refers to one or more of such compounds, while “the enzyme” includes a particular enzyme as well as other family members and equivalents thereof as known to those skilled in the art.

RNA stabilizing sequence: is an inserted sequence of from about 2 to 25 nucleotides, usually from about 5 to 15 nucleotides at the 3′ region of an RNA that acts to protect the RNA from degradation by 3′ to 5′ exonucleases. The stabilizing sequence is a homopolymer, where a poly (G) tract is preferred, although benefits are obtained from insertion of a poly (U) sequence, and in some instances with a poly (C) tract. The preferred location for the stabilizing sequence is at the 3′ terminus of the RNA.

The RNA stabilizing sequence may be chemically synthesized by techniques well-known in the art, for example, by using an automated synthesizer such as the Applied Biosystems 380A automated synthesizer, and ligated to the RNA. Alternatively and preferably, the RNA stabilizing sequence is inserted by transcription from a promoter in vitro or in vivo, where the stabilizing sequence is provided by a DNA template for transcription. The template for transcription may be a plasmid or other vector, where the complementary sequence to the stabilizing sequence is inserted by conventional recombinant techniques at the appropriate position relative to the transcription unit.

The stabilizing sequence may alternatively be included in the DNA transcription template by providing amplification primers containing the stabilizing sequence or complement thereof. When the targeted nucleic acid is then amplified, it will result in a DNA template comprising a stabilizing sequence.

Transcription enhancing sequence. For efficient transcription from linear DNA molecules, such as amplification products, restriction fragments, etc., it has been found that a phage derived RNA polymerase can require one additional nucleotide to be present 5′ to the promoter sequence, preferably at least two nucleotides, and more preferably at least about 5 nucleotides. Phage derived RNA polymerases include T7 polymerase, SP6 polymerase, T4 polymerase, etc. T7 polymerase is of particular interest. The base composition of the transcription enhancing sequence does not affect it's performance. Promoter sequences for these enzymes are well known in the art.

The transcription enhancing sequence may be inserted by convention recombinant techniques into a DNA, e.g. a plasmid or other vector, such that on release of a linear fragment from the vector for transcription, there is included at least about 5 or more nucleotides present at the terminus 5′ to the promoter.

Preferably, the transcription enhancing sequence is used in combination with an amplification reaction, such as PCR, where the promoter and transcription enhancing sequence, or complement thereof, is provided in an amplification primer. For use in such primers, the transcription enhancing sequence will generally be from about 5 nucleotides and may extend to the length of the primer, and may be provided as a random sequence, or to provide a desired functionality, e.g. as a tagged sequence, an enzyme recognition sequence, etc. The PCR primer may be specific for a targeted sequence, or may be generic, or universal primers designed to amplify all sequences present in a complex mixture.

Expression constructs: The mRNA to be expressed in the methods of the invention will have the general characteristics of an expressed sequence, which include an open reading frame, appropriate signals for the start of translation, and generally will also include signals for the termination of translation. As described above, the sequence of the mRNA for expression is optionally derived from a cloned DNA sequence, e.g. a coding sequence present on a vector.

Vectors include, for example segments of chromosomal, non-chromosomal and synthetic DNA sequences, such as various bacterial plasmids, e.g. plasmids from E. coli including col E1, pCR1, pBR322, pMB9 and their derivatives; wider host range plasmids, e.g. RP4; phage DNAs, e.g. derivatives of phage λ, other DNA phages, e.g. M13 and filamentous single stranded DNA phages, and vectors derived from combinations of plasmids and phage DNAs, such as plasmids which have been modified to employ phage DNA or other expression control sequences. Notwithstanding the use of such vectors, the invention also allows use of non-cloned genomic DNA; mRNA from primary sources, and cDNA as a template for transcription.

Where the stabilized mRNA is transcribed from a vector sequence, as described above, the coding sequence is linked to regulatory sequences as appropriate to obtain the desired expression properties. These can include promoters attached either at the 5′ end of the sense strand or at the 3′ end of the antisense strand, enhancers, terminators, operators, repressors, and inducers. The promoters can be regulated or constitutive. In some situations it may be desirable to use conditionally active promoters. These are linked to the desired nucleotide sequence using the techniques described above for linkage to vectors. Any techniques known in the art can be used.

Within each specific cloning or expression vector, various sites may be selected for insertion of the DNA sequences of this invention. These sites are well recognized by those of skill in the art. Various methods for inserting DNA sequences into these sites to form recombinant DNA molecules are also well known. These include, for example, dG-dC or dA-dT tailing, direct ligation, synthetic linkers, exonuclease and polymerase-linked repair reactions followed by ligation, or extension of the DNA strand with DNA polymerase and an appropriate single-stranded template followed by ligation. It is to be understood that a cloning or expression vehicle useful in this invention need not have a restriction endonuclease site for insertion of the chosen DNA fragment. Instead, the vehicle could be joined to the fragment by alternative means.

The vector or expression vehicle, and, in particular, the sites chosen therein for insertion of the selected DNA fragment and the expression control sequences employed in this invention are determined by a variety of factors, e.g. number of sites susceptible to a particular restriction enzyme, size of the protein to be expressed, expression characteristics such as the location of start and stop codons relative to the vector sequences, and other factors recognized by those of skill in the art. The choice of a vector, expression control sequence, and insertion site for a desired protein sequence is determined by a balance of these factors, not all selections being equally effective for a given case.

Useful promoters include T4 promoters, the lac system, the trp system, the TAC or TRC system, the major promoter regions of phage λ, the control regions of fd coat protein, the promoter for 3-phosphoglycerate kinase or other glycolytic enzymes, the promoters of acid phosphatase, e.g. Pho5, and other sequences known to control the expression of genes of prokaryotic cells or their viruses, and various combinations thereof. Eukaryotic promoters, as known in the art, may also be used.

In one embodiment of the invention, an expression vector is provided, comprising a transcriptional promoter; sequences suitable for insertion of an open reading frame, e.g. a polylinker with multiple restriction sites, a unique restriction site, i.e. a site that is present in the vector only once; a transcription terminator sequence; and an mRNA stabilizing sequence.

In vitro amplification: The term “amplification primer”, as used above, refers to an oligonucleotide which acts to initiate synthesis of a complementary DNA strand when placed under conditions in which synthesis of a primer extension product is induced, i.e., in the presence of nucleotides and a polymerization-inducing agent such as a DNA-dependent DNA polymerase and at suitable temperature, pH, metal concentration, and salt concentration. As described above, amplification primers may be used to insert the RNA stabilization sequence, and/or the transcription enhancing sequence in combination with a phage specific promoter, to generate a DNA template for transcription of the RNA(s) of interest. Target sequences may be a single sequence or a pool of sequences. Preferably, amplification primers are from 30 to 50 nucleotide long and have Tm's between 80° C. and 120° C. Preferably, such amplification primers are employed with a first annealing temperature of between about 72° C. to about 84° C. Preferably, annealing temperatures are selected to ensure specificity in amplification and detection. Typically, annealing temperatures are selected in the range of from 1-2° C. above or below the melting temperature of an amplification primer to about 5-10° C. below such temperature. Guidance for selecting appropriate primers can be found in many references.

Amplification primers are readily synthesized by standard techniques, e.g., solid phase synthesis via phosphoramidite chemistry, as disclosed in U.S. Pat. Nos. 4,458,066 and 4,415,732 to Caruthers et al; Beaucage et al. (1992) Tetrahedron 48:2223-2311.

The PCR method for amplifying target polynucleotides in a sample is well known in the art and has been described by Saiki et al. (1986) Nature 324:163, as well as by Mullis in U.S. Pat. No. 4,683,195, Mullis et al. in U.S. Pat. No. 4,683,202, Gelfand et al. in U.S. Pat. No. 4,889,818, Innis et al. (eds.) PCR Protocols (Academic Press, NY 1990), and Taylor (1991) Polymerase chain reaction: basic principles and automation, in PCR: A Practical Approach, McPherson et al. (eds.) IRL Press, Oxford.

Briefly, the PCR technique involves preparation of oligonucleotide primers that flank the target nucleotide sequence to be amplified, and are oriented such that their 3′ ends face each other, each primer extending toward the other. The polynucleotide sample is extracted and denatured, preferably by heat, and hybridized with the primers which are present in molar excess. Polymerization is catalyzed in the presence of deoxyribonucleotide triphosphates (dNTPs). This results in two “long products” which contain the respective primers at their 5′ ends covalently linked to the newly synthesized complements of the original strands. The reaction mixture is then returned to polymerizing conditions, e.g., by lowering the temperature, inactivating a denaturing agent, or adding more polymerase, and a second cycle is initiated. The second cycle provides the two original strands, the two long products from the first cycle, two new long products replicated from the original strands, and two “short products” replicated from the long products. The short products have the sequence of the target sequence with a primer at each end. On each additional cycle, an additional two long products are produced, and a number of short products equal to the number of long and short products remaining at the end of the previous cycle. Thus, the number of short products containing the target sequence grow exponentially with each cycle. Preferably, PCR is carried out with a commercially available thermal cycler.

PCR amplification is carried out by contacting the sample with a composition containing first and second primers, sufficient quantities of the four deoxyribonucleotide triphosphates (dATP, dGTP, dCTP and dTTP) to effect the desired degree of sequence amplification, and a primer- and template dependent polynucleotide polymerizing agent, such as any enzyme capable of producing primer extension products, for example thermostable DNA polymerases isolated from Thermus aquaticus (Taq), which is available from a variety of sources, Thermus thermophilus, Bacillus stereothermophilus, or Thermococcus litoralis, and the like.

In one embodiment of the invention, mRNA from a cell source of interest is reverse transcribed into cDNA, where the cDNA may then be used as the template for PCR. The technique if commonly referred to as RT-PCR (see Kawasaki et al. (1991) Amplification of RNA. In PCR Protocols, a Guide to Methods and Applications, Innis et al. eds., (Academic Press, Inc., San Diego) pp. 21-27). This embodiment finds particular use for the synthesis of proteins from eukaryotic mRNA, particularly mRNA from primary cells.

In Vitro Transcription: as used herein refers to the cell-free transcription of RNA from DNA or RNA templates utilizing a reaction mixture comprised of cellular extracts and other biological components necessary for creating the transcription machinery. Coupling of in vitro transcription and translation reactions are also known in the art. Efficient in vitro transcription systems of particular interest use of phage specific polymerases, e.g. T7, T4, SP6, and T3 polymerases. These transcription reaction mixtures may be combined with translation extracts, e.g. from E. coli extracts; wheat germ; rabbit reticulocytes; etc.

Of particular interest is the transcription system using bacteriophage T7 polymerase (see U.S. Pat. No. 5,869,320, Studier et al.) Materials required for an in vitro transcription system include a buffer of suitable pH, salts, magnesium ions, T7 RNA polymerase, NTPs (which include ATPs to drive the transcription reaction and nucleotides to be incorporated into mRNA transcripts), and a DNA template with T7 promoter. Salts such as sodium chloride maintain an reducing environment that is favorable to the transcription process. It is known in the art that the presence of organic bases such as spermidine, 1,8-octanediamine, cadaverine, and agmatine, and ethylated polyamine analogues such as 1,8-bis(ethylamino)octane and 1,5-bis (ethylamino)pentane can greatly enhance in vitro transcription via binding to T7 RNA polymerase. (Iwata et al. (2000) Bioorg. Med. Chem. 8(8):2185-94.) The use of this type of polyamine serves to improve mRNA transcription sequence and size fidelity. It will be understood by one of skill in the art that substitutes with the same functional properties may be readily used.

In vitro translation: as used herein refers to the cell-free synthesis of proteins or peptides in a reaction mix comprising biological extracts and/or defined reagents. The reaction mix will comprise at least ATP, an energy source; mRNA; amino acids; enzymes and other reagents that are necessary for the synthesis, e.g. ribosomes, tRNA, polymerases, transcriptional factors, etc. Also included are enzyme(s), where indicated. Such enzymes may be present in the extracts used for translation, or may be added to the reaction mix. Such synthetic reaction systems are well-known in the art, and have been described in the literature. The cell free synthesis reaction may be performed as batch, continuous flow, or semi-continuous flow, as known in the art.

The reactions may utilize a large scale reactor, small scale, or may be multiplexed to perform a plurality of simultaneous syntheses. Continuous reactions will use a feed mechanism to introduce a flow of reagents, and may isolate the end-product as part of the process. Batch systems are also of interest, where additional reagents may be introduced to prolong the period of time for active synthesis. A reactor may be run in any mode such as batch, extended batch, semi-batch, semi-continuous, fed-batch and continuous, and which will be selected in accordance with the application purpose.

Translation may be coupled to in vitro synthesis of mRNA from a DNA template. Such a cell-free system will contain all factors required for the translation of mRNA, for example ribosomes, amino acids, tRNAs, aminoacyl synthetases, elongation factors and initiation factors. Materials for protein synthesis may include salt, polymeric compounds, cyclic AMP, inhibitors for protein or nucleic acid degrading enzymes, inhibitors or regulators of protein synthesis, oxidation/reduction adjusters, non-denaturing surfactants, buffer components, spermine, spermidine, etc.

The salts preferably include potassium, magnesium, ammonium and manganese salt of acetic acid or sulfuric acid, and some of these may have an amino acid as a counter anion. The polymeric compounds may be polyethylene glycol, dextran, diethyl aminoethyl, quaternary aminoethyl and aminoethyl. The oxidation/reduction adjuster may be dithiothreitol, ascorbic acid, glutathione and/or their oxides. Also, a non-denaturing surfactant such as Triton X-100 may be used at a concentration of 0-0.5 M. Spermine and spermidine may be used for improving protein synthetic ability, and cAMP may be used as a gene expression regulator.

When changing the concentration of a particular component of the reaction medium, that of another component may be changed accordingly. For example, the concentrations of several components such as nucleotides and energy source compounds may be simultaneously controlled in accordance with the change in those of other components. Also, the concentration levels of components in the reactor may be varied over time.

Preferably, the reaction is maintained in the range of pH 5-10 and a temperature of 20°-50° C., and more preferably, in the range of pH 6-9 and a temperature of 25°-40° C.

When using a protein isolating means in a continuous operation mode, the product output from the reactor through a membrane flows into the protein isolating means. In a semi-continuous operation mode, the outside or outer surface of the membrane is put into contact with predetermined solutions that are cyclically changed in a predetermined order. These solutions contain substrates such as amino acids and nucleotides. At this time, the reactor is operated in dialysis, diafiltration batch or fed-batch mode. A feed solution may be supplied to the reactor through the same membrane or a separate injection unit. Synthesized protein is accumulated in the reactor, and then is isolated and purified according to the usual method for protein purification after completion of the system operation.

Where there is a flow of reagents, the direction of liquid flow can be perpendicular and/or tangential to a membrane. Tangential flow is effective for recycling ATP and for preventing membrane plugging and may be superimposed on perpendicular flow. Flow perpendicular to the membrane may be caused or effected by a positive pressure pump or a vacuum suction pump. The solution in contact with the outside surface of the membrane may be cyclically changed, and may be in a steady tangential flow with respect to the membrane. The reactor may be stirred internally or externally by proper agitation means.

During protein synthesis in the reactor, the protein isolating means for selectively isolating the desired protein may include a unit packed with particles coated with antibody molecules or other molecules immobilized with a component for adsorbing the synthesized, desired protein, and a membrane with pores of proper sizes. Preferably, the protein isolating means comprises two columns for alternating use.

The amount of protein produced in a translation reaction can be measured in various fashions. One method relies on the availability of an assay which measures the activity of the particular protein being translated. An example of an assay for measuring protein activity is a luciferase assay system, or chloramphenicol acetyl transferase assay system. These assays measure the amount of functionally active protein produced from the translation reaction. Activity assays will not measure full length protein that is inactive due to improper protein folding or lack of other post translational modifications necessary for protein activity.

Another method of measuring the amount of protein produced in coupled in vitro transcription and translation reactions is to perform the reactions using a known quantity of radiolabeled amino acid such as ³⁵S-methionine or ³H-leucine and subsequently measuring the amount of radiolabeled amino acid incorporated into the newly translated protein. Incorporation assays will measure the amount of radiolabeled amino acids in all proteins produced in an in vitro translation reaction including truncated protein products. The radiolabeled protein may be further separated on a protein gel, and by autoradiography confirmed that the product is the proper size and that secondary protein products have not been produced.

### Methods of Use

The methods of the present invention find use in the enhanced production of polypeptides in vitro, e.g. for bulk production of a desired protein, for high throughput production of multiple proteins simultaneously, etc. It is not necessary for the sequence of interest to be cloned, as amplification reactions can be used to provide a homogenous DNA template for transcription reactions.

In other aspects of the invention, the methods may be used to produce synthesize polypeptides in vitro for identification of a protein species encoded by a gene or genes of interest. In conventional proteomics methods a series of individual spots from 2-D protein gels or individual species separated by other means are applied to MALDI-TOF mass spectroscopy, microsequencing, or other methods to obtain the composition of the protein and thereby infer the sequence of the gene encoding the protein located at that position. The protein synthesis procedure described in herein allows one to directly identify the spot containing the protein encoded by a gene of interest.

This is done by using RT-PCR (for products of eukaryotic cells) or simply PCR (for species having no introns) to amplify the desired template and install a T7 or other highly efficient promoter, preferably with a transcription enhancing sequence; and an RNA stabilization sequence at the 5′ and 3′ ends of the template respectively. The protein is then synthesized in vitro and may be analyzed directly, or may be added to an extract from the cell of interest. Controls in which the protein of interest has not been produced are also prepared. Analysis of the experimental and control mixtures by 2-D gel analysis or other fractionation/separation procedures identifies the over-produced or labeled species and shows its position in the gel. Thus, one can directly identify proteins in gels that correspond to a gene of interest and subsequently follow the expression of this protein, rather than carry out analysis of multiple proteins isolated from gels in order to identify the position of the one that corresponds to the predicted product of the gene of interest.

In another embodiment an epitope tag is added to one or more proteins of interest, by insertion of a tag sequence during the amplification reaction, or by cloning into a recombinant vector. Where multiple proteins are tagged, different epitope tags may be used. The newly synthesized protein may then be isolated by virtue of the epitope tag from the synthetic reaction; and used to obtain other proteins in those extracts or other preparations that are capable of binding to the target protein.

Multiple proteins may be made in vitro from open reading frames of genomic DNA sequences, where the sequences are ligated or amplified with primers comprising the RNA stabilization sequence; and a promoter/transcription enhancing sequence. Such proteins find use in analysis of protein/protein interactions, small molecule screening assays, etc. The invention allows high throughput screening of protein/protein interactions.

It should be understood that the DNA sequences that code for the desired polypeptides may include nucleotides which are not part of an actual gene coding for the particular polypeptide, for example in the construction of targeted mutations, including deletions, additions, etc. For example, a DNA sequence may be fused in the same reading frame to a portion of a DNA sequence coding for at least one eukaryotic or prokaryotic carrier protein, or to a DNA sequence coding for at least one eukaryotic or prokaryotic signal sequence, or combinations thereof. Such constructions may aid in expression of the desired DNA sequence, improve purification etc. The DNA sequence may alternatively include an ATG start codon, alone or together with other codons, fused directly to the sequence encoding the first amino acid of a desired polypeptide. Such constructions enable the production of, for example, a methionyl or other peptidyl polypeptide. This N-terminal methionine or peptide may then, if desired, be cleaved intra- or extra-cellularly by a variety of known processes or the polypeptide used together with the methionine or other fusion attached to it in various compositions and methods.

It is to be understood that this invention is not limited to the particular methodology, protocols, cell lines, animal species or genera, constructs, and reagents described, as such may, of course, vary. It is also to be understood that the terminology used herein is for the purpose of describing particular embodiments only, and is not intended to limit the scope of the present invention which will be limited only by the appended claims.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the cell lines, constructs, and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### EXPERIMENTAL

### EXAMPLE 1

### Effects of 3′ Terminus Modifications on Translation and Functional Decay of E. coli mRNA in vitro

Experimental Procedures

Strains and Plasmids: E. coli B strain BL21 (F, hsdS, gal, OmpT⁻) was initially used for S-30 preparation when mRNAs (CAT and lacZα) were used in vitro translation. SL119, a recD derivative of BL21 was used to prepare S-30 for a coupled transcription/translation system. For preparation of 30S ribosomal subunits, E. coli K12 strain CA244 (lacZ, trp, relA, spoT) was used. All plasmids were maintained in E. coli DH5α (supE44, hsdR17, recA1, endA1, gyrA96, thi-1, (laclZYA-argF)U169, deoR (φ80dlac (lacZ)M15). pET3aα was constructed by amplifying LacZα fragment (amino acids 1-94) from chromosomal DNA of E. coli strain N3433 (lacZ43, relA, spoT, thi-1) using oligonucleotides 5′α (SEQ ID NO: 1) (5′-ACAGGATCCATGACCATGATTACGGAT) and 3′α (SEQ ID NO: 2) (5′-ACAGGATCCGTGCATCTGCCAGTTTGA) and cloning into the BamHI site of pET3a (Novagen). pET3a-CAT was constructed by amplifying CAT gene from pACYC184 using oligonucleotides 5′ CAT (SEQ ID NO: 3) (5′-ACAGGATCCAGGAGGCTCGAGATGGAGAAAAAAATCACTGGA) and (SEQ ID NO: 4) 3′CAT (5′ACAGGATCCTTACGCCCCGCCCTGCCACTC) and cloning into the BamHI site of pET3a. Plasmid pGL3Basic was purchased from Promega and pJSE371 was a gift.

Enzymes and Reagents: AMV reverse transcriptase, T4 polynucleotide kinase (T4 PNK), T7 RNA polymerase and restriction enzymes were from New England Biolabs. Oligonucleotides were from Life Technology Inc. [γ³²P]-ATP (6,000 Ci/mmol), [α³²P]-UTP (6,000 Ci/mmol), [³H]-chloramphenicol (38.9 Ci/mmol), and the Renaissance ECL detection kit were from NEN Life Science Products. Anti T7·tag antibody HRP conjugate and T7·tag affinity purification kit were from Novagen. M2 antibody was from Kodak and anti rabbit IgG conjugated with HRP was from Promega. Polyclonal antibodies against E. coli PNPase was a gift. Other chemicals and tRNAs were purchased from Sigma.

S-30 Preparation and Reactions: An E. coli coupled transcription/translation system (S-30) was prepared from E. coli strain BL21 essentially as described by Lesley et al. (1991) Journal of Biological Chemistry 266(4), 2632-8. mRNAs containing 20A tails were used to determine the optimal concentration of CAT mRNA and lacZα mRNA in reactions. Optimal protein production was observed at 120 nM mRNA for both mRNAs. Coupled transcription/translation reactions were incubated at 37° C. for one hour in reaction mixtures containing agarose gel purified DNA (50 ng/μl), unless otherwise indicated.

Synthesis of DNA and RNA: All mRNAs used here were synthesized using MEGAscript™ T7 kit (Ambion) and PCR DNA as template according to manufacturer's instructions. RNA was purified from 6% acrylamide gel containing 8 M urea. For in vitro synthesis of CAT and lacZα mRNAs containing no 3′ additions or containing A20, A40, or G20 in vitro, PCR-generated DNAs were prepared using 5′ primer (SEQ ID NO: 5) (5′ TAATACGACTCACTATAGG) and 3′ primer (SEQ ID NO: 6) (5′ (none, 20C, 20T, or 40T) AAGGCTGTTAGCAGCCGGATCC) and pET3a-α or pET3a-CAT as template. PCR DNAs for coupled transcription/translation reactions were prepared as follows: (A) For PCR DNAs containing CAT coding region, a 5′ primer which installs the T7 promoter with different length of extra base-pairs upstream of the T7 promoter and 3′ primer which installs 3 ′ tails were used to amplify the CAT coding region from pET3a-CAT. The 5′ primer was (SEQ ID NO: 7) 5′TAATACGACTCACTATAGG with extra base-pairs at the 5′ which were either 20 random nucleotides (20N) or different lengths of upstream sequence of T7 promoter present in pET3a-CAT plasmid (SEQ ID NO: 8) (5′AGATCTCGATCCCGCGAAAT) and the 3′ primer was 5′-complementary sequence of tails (SEQ ID NO: 9) -TTACGCCCCGCCCTGCCA (stop codon is in bold type). (B) For PCR DNAs containing fire fly luciferase gene, 5′ primer was (SEQ ID NO: 10) 5′GAAATTAATACGACTCACTATAGG GTTAACTTTMGAA GGAGCC ACCATGG AAGACGCCA (the consensus T7 promoter is underlined and start codon is in bold type), 3′ primers were 5′-complementary sequence of tails (SEQ ID NO: 11) -TTAC ACGGCG ATCT TTCCGCC (stop codon is in bold type), and template was pGL3Basic. (C) For PCR DNAs containing guanosine pentaphosphate synthetase I (GPSI), 5′ primer was (SEQ ID NO: 12) 5′ GAAATTAATACGACTCACTATAGG (the consensus T7 promoter is underlined), 3′ primers was 5′-complementary sequence of tails- (SEQ ID NO: 13) TACGGGACGTCACTGCTC (stop codon is in bold type), and template was pJSE371. (D) Coding region of telomere binding protein (TP, Bao and Cohen, manuscript in preparation) was amplified from Streptomyces rochei chromosome using the 5′ primer, (SEQ ID NO: 14) 5′GAAATTAATACGACTCACTATAGGGTTAACTTTAAGAAGGAGATATACATATGGTGGAC TCGATCGGAGACGG (the consensus T7 promoter is underlined and start codon is in bold type) and the 3′ primer, 5′-complementary sequence of tails-(SEQ ID NO: 15) CTACTT GT CGTCATCGTCCTTGTAGTCCAGCTGGATCTCGATCTG. (The stop codon is in bold type and the sequence for the Flag•tag is underlined.)

Preparation of 30S ribosomal subunits: 30S ribosomal subunits were prepared essentially as described by Moazed and Noller except that frozen E. coli CA244 cells were opened by passing them through French Press at 10,000 p.s.i. twice and 70S ribosome pellet was washed and resuspended twice in buffer B before being dialyzed against buffer C. S1 depleted 30S ribosomal subunits were prepared by the procedure published by Tal et al. (19). Briefly, purified 30S was dialyzed against a low strength buffer (1 mM Tris-HCl, pH 7.5) followed by precipitation of S1-depleted 30S. S1 protein is the largest of all 30S ribosomal proteins and removal of S1 protein in 30S was confirmed by visualizing 30S subunit ribosomal proteins in SDS PAGE.

Extension Inhibition (Toeprinting) Assay: Toeprinting assays were performed as described by L. Gold et al. using AMV reverse transcriptase at one unit per reaction. The primers CAT-TP (SEQ ID NO: 16) (5′GGATCCGCGACCCATTTG) and α-TP (SEQ ID NO: 17) (5′GGGTTTTCCCAGTCACGA) which are complementary to CAT and lacZα transcripts, respectively, were 5′ end-labeled with [γ³²P]-ATP and T4 PNK and purified from 15% acrylamide containing 8 M urea. We determined optimal conditions for binding of the 30S ribosomal subunits to mRNA using reverse transcriptase amounts ranging from 0 to 1.6 units per reaction and mRNA to primer ratios from 0.25 to 1 and then tested CAT and lacZα mRNAs in this assay.

Chloramphenicol acetyltransferase assays: CAT activity was determined as described by Nielsen et al. (1989) Analytical Biochemistry 179(1), 19-23.

Luciferase assay: Luciferase assay was performed according to manufacturer's instructions and the enzymatic activity was measured in TD-20e Luminometer (Turner).

Protein Work and Western Blotting: CAT protein was purified from BL21 (DE3) harboring pET3a-CAT after one hour induction with 1 mM IPTG using T7·tag purification kit according to manufacturer's instructions. The protein concentrations were estimated using Coomassie Brillant Blue G250as described by Sedmak and Grossberg (22), using bovine serum albumin as a standard. Prestained protein molecular weight standards from Life TechnologiesInc. were used as size markers.

Proteins were run on a 10% Tricine-SDS-PAGEas described by Hermann and von Jagow (23) and gels were electroblotted to a nitrocellulose filter and probed as described by Hagege and Cohen (1997) Molecular microbiology 25(6), 1077-1090. The dilutions used for antibodies were 1:10,000 for anti-T7·tag-HRP, 1:1,000 for anti-Flag (M2), and 1:5,000 for anti-PNPase, anti mouse IgG-HRP, and anti rabbit IgG-HRP. When blots were used for reprobing they were stripped at 50° C. for 30 minutes with occasional agitation in stripping buffer (100 mM 2-mercaptoethanol, 2% SDS, 62.5 mM Tris-HCl, pH6.7), followed by two times wash in TBS-T (Tris-buffer saline-0.05% Tween 20) for 20 minutes.

Results

Effects of polynucleotide tails on translation of transcripts in vitro: In E. coli, translation begins on nascent mRNA during the course of its synthesis, so that any translational enhancement by 3′ poly(A) additions would necessarily be restricted to subsequent cycles of translation of already completed transcripts. Accordingly, we tested the ability of full length transcripts containing or lacking poly(A) tails to generate protein in vitro. Transcripts encoding chloramphenicol acetyl transferase (CAT) or the alpha (α) fragment of the β-galactosidase (LacZ) protein uniformly labeled with ³²P-UTP were synthesized in vitro by bacteriophage T7 RNA polymerase using PCR-generated DNA fragments as template (see EXPERIMENTAL PROCEDURES). The transcripts, which contained 0, 20, or 40 adenosine (A) residues at the 3′ end were gel purified and added to an E. coli extract-based reaction mix for in vitro translation. Transcripts lacking homopolymer tails or containing twenty 3′ guanosine (G) residues were included as controls. The mRNAs chosen for translation were relatively small (417 and 807 nucleotides for lacZα and CAT respectively) and the proteins they encode are well characterized.

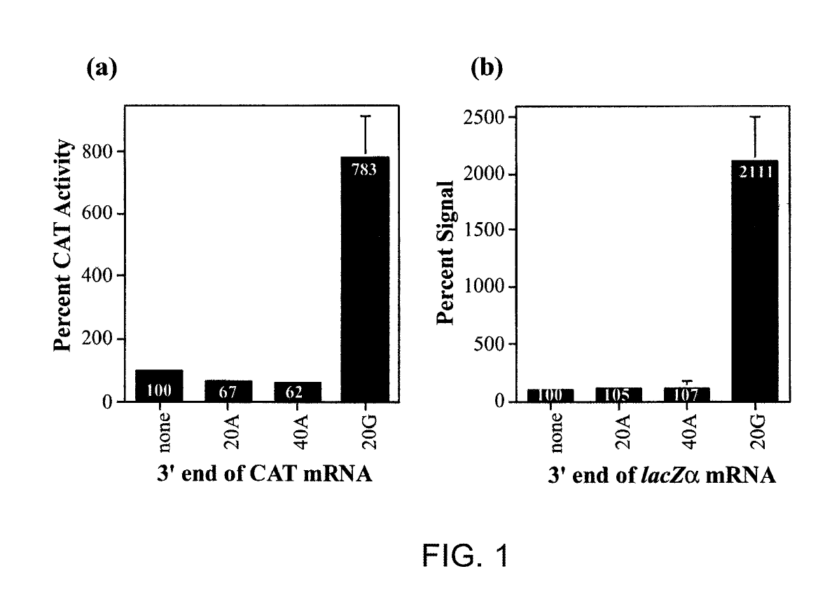

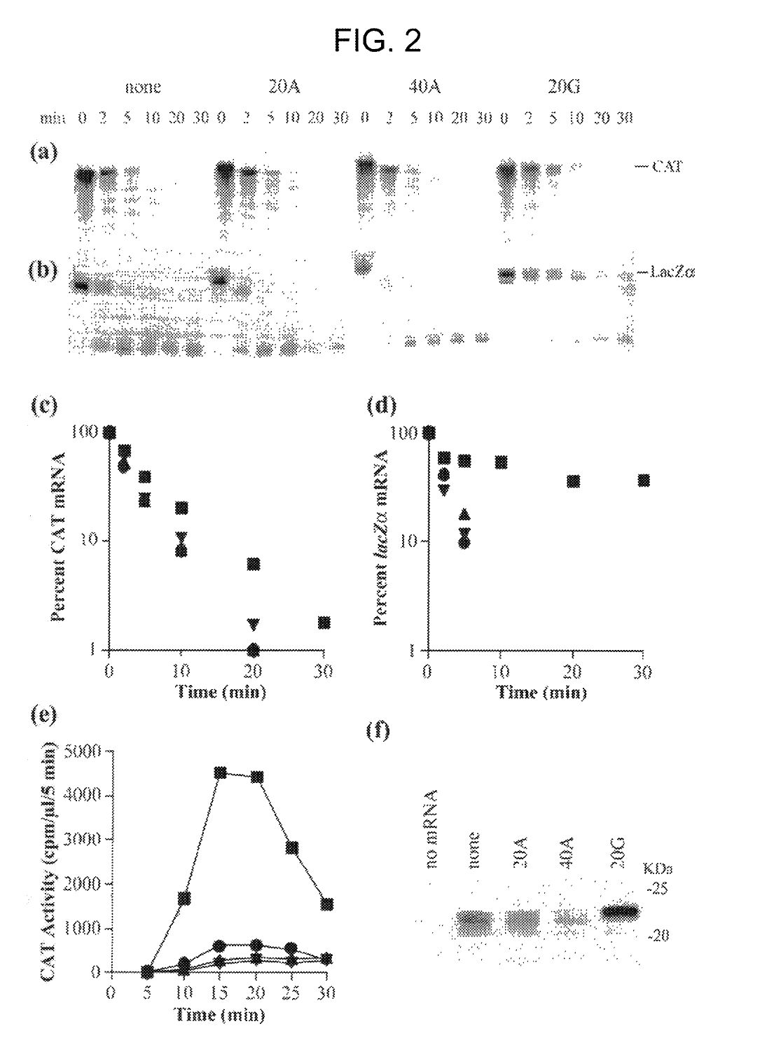

Testing of these mRNAs to yield protein in in vitro translation reactions revealed no detectable translational effects of polyadenylation on CAT and LacZα production (FIG. 1, A and B). Poly(A) tails 20 or 40 nucleotides in length were removed from mRNA molecules added to reaction mixtures by two minutes, leading to decay of primary transcripts at the same rate as non-adenylated RNA(FIG. 2, A-D). These tails had no detectable effect on mRNA chemical half life in our in vitro protein synthesis reaction mixtures, as determined by measuring the percent of full-length mRNA remaining in the reaction (FIG. 2, A-D). However, in contrast to the effects of poly(A) tails, addition of poly(G) tails to these transcripts resulted in dramatically-enhanced production of CAT and LacZα protein in vitro (FIG. 1, A and B).

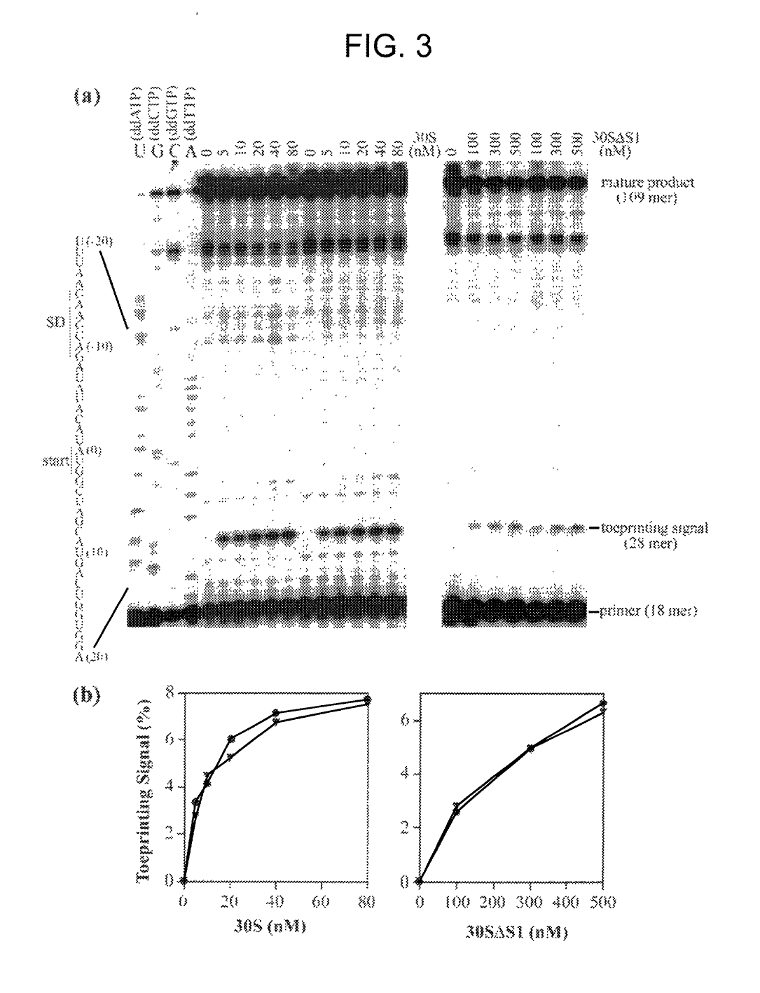

Further analysis showed that poly(G) tail-mediated stimulation of protein synthesis during in vitro translation of CAT and lacZα transcripts resulted from retarded degradation of transcripts and their markedly enhanced ability to promote protein synthesis. Moreover, as seen in FIG. 1 and FIG. 2, C and D, the effect of poly(G) tails on the translation of CAT and lacZα mRNAs was about 4 to 6 times greater than their effect on mRNA decay, as measured by Northern blotting, suggesting that 3α poly(G) stretches increase mRNA functional half life in E. coli cell extracts to a greater extent than chemical half life. This notion was confirmed by quantitation of biochemically-active CAT protein produced by mRNA incubated with E. coli cell extracts for various lengths of time (FIG. 2e). Western blot analysis of N-terminal T7 epitope-tagged CAT proteins encoded by mRNAs that were either not ‘tailed’ or alternatively were polyadenylated or polyguanylated, showed that primary transcripts lacking tails produced substantial amounts of CAT protein that was slightly shorter in length than the CAT protein produced by polyguanylated transcripts (FIG. 2F). This result, together with evidence that transcripts lacking tails generated much less CAT biochemical activity than polyguanylated transcripts (FIG. 1), suggested that inactive C-terminally-truncated CAT proteins were being produced in our reaction mixtures by 3′ terminally truncated mRNA decay intermediates—and consequently that degradation of, and functional inactivation of CAT transcripts was proceeding from the 3′ mRNA end. This finding was surprising, as the initial step in E. coli mRNA inactivation is believed to be endonucleolytic cleavage rather than by 3′ to 5′ exonucleolytic decay.

Whereas poly(A) tails were rapidly removed from transcripts during incubation in vitro in protein synthesis reaction mixtures, we found that primary transcripts and their 3′ poly(A) additions were not detectably degraded over a 30 minute period in vitro in toeprinting assay mixtures that used highly purified ribosomes and reverse transcriptase. We therefore could use a toeprinting assay, which measures the rate of formation of translation initiation complexes between mRNA and 30S ribosomal subunits, to test for possible translational enhancement by poly(A) tails, as initiation is known to be the rate limiting step in mRNA translation. Toeprinting signals, which result from mRNA bound to 30S ribosomal subunits were located at a distance of 15 nucleotides from the 5′-most nucleotide of the start codon of CAT mRNA (FIG. 3A). This agrees well with earlier evidence that the site of toeprinting usually occurs 15 nucleotides from the first nucleotide of the start codon. CAT mRNAs with no tails or with tails consisting of 40As showed similar binding efficiency to 30S ribosomal subunits (FIG. 3B). Poly(A) tailed mRNAs and untailed primary transcripts were further tested for the efficiency of binding to ribosomal protein 30S ribosomal subunits depleted of S1 (30S-S1) (see EXPERIMENTAL PROCEDURES), as it has been speculated that S1 may play a role in translation by recruiting 30S to poly(A) tailed mRNAs. As previously shown, the binding efficiency decreased by about 25-fold when 30S-S1 was used in reactions. However, there was no difference in binding efficiency for poly(A) tailed mRNA (FIG. 3).

Insertion of 5′ base pairs promotes transcription by T7 RNA polymerase: The ability of poly(G) tails to significantly protect mRNA from functional decay during in vitro protein synthesis suggested that the installation of poly(G) tails onto mRNA molecules might be useful as a general strategy for increasing protein yield in vitro. If so, we hypothesized, poly(G)-tailed transcripts made by in vitro transcription of PCR-generated templates containing genomic open reading frames (ORFs) potentially could facilitate the synthesis of proteins in vitroin coupled transcription/translation reactions. The approach we devised to test this notion involved the synthesis of run-off CAT gene transcripts by the highly efficient bacteriophage T7 RNA polymerase. The 5′ primer used to generate the template by PCR installs the bacteriophage T7 promoter near the 5′ end of the template and the 3′ primer installs a 20 nucleotide stretch of poly(C) at the template's 3′ end; this was expected to lead to synthesis of homopolymeric G tails on T7-generated transcripts. The E. coli cell extracts employed for these experiments were prepared from an exodeoxyribonuclease deficient (recD) strain (SL119) to minimize degradation of linear DNA templates.

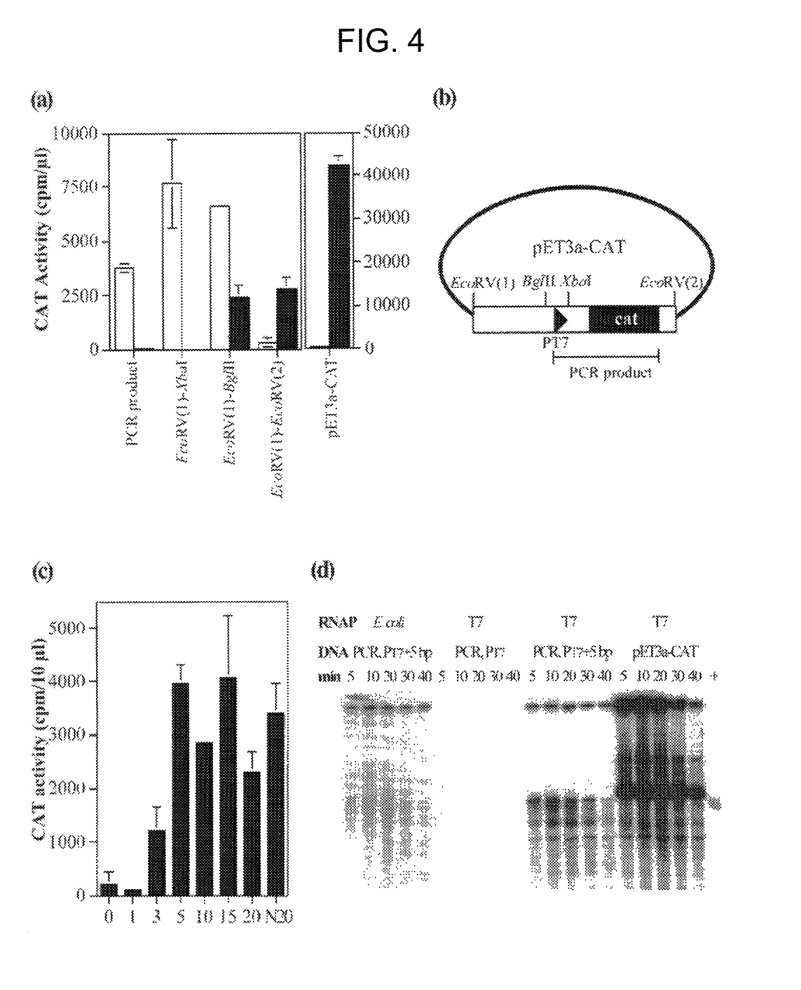

Using coupled transcription/translation, no chemical or functional stabilization of CAT mRNA synthesized as described above was observed. This contrasted with what had been seen when poly(G) tailed transcripts synthesized in vitro by T7 RNA polymerase were used as template, raising the possibility that synthesis of poly(G) tailed transcripts by T7 RNAP was not occurring in these E. coli cell extracts. Consistent with this notion, transcription of PCR-generated DNA fragments containing the T7 promoter has previously been reported to be mediated by the E. coli RNAP rather than the T7 RNAP in coupled transcription/translation reaction mixtures. If transcription was in fact being carried out by the E. coli RNAP in our reaction mixtures, the inability of the E. coli enzyme to efficiently transcribe homopolymer sequences potentially could lead to the absence of poly(G) tails on transcripts. This interpretation was tested and confirmed by the finding that addition of rifampicin, an inhibitor of E. coli RNAP but not of T7 RNAP, to reaction mixtures sharply decreased protein production in vitro (FIG. 4a).

In contrast with our results and those of Lesley et al. (1991) Journal of Biological Chemistry 266(4), 2632-8, Nevin and Pratt (1991) Febs Letters 291(2), 259-63 observed that linearized DNA containing the T7 promoter was efficiently transcribed in E. coli cell extracts in the presence of rifampicin. Comparison of the sequences of our template with the one used by Nevin and Pratt indicated that their DNA template contained additional base-pairs 5′ to the T7 promoter. To test the notion that nucleotides 5′ to the T7 promoter are crucial to the ability of the T7 RNAP to initiate transcription on linear DNA templates, DNA fragments prepared from restriction enzyme digested pET3a-CAT plasmid, as shown in FIG. 4B, were used as substrates for coupled transcription/translation assays in vitro. The results of these experiments (FIG. 4A) showed that rifampicin-independent transcription occurred on a template containing 17 base pairs 5′ to the T7 promoter (i.e., the restriction endonuclease-generated BgIII-EcoRV DNA fragment), but that a PCR-generated DNA fragment that included the same promoter but lacked additional upstream base pairs failed to function as a template for T7 RNAP. We also observed in these experiments that CAT protein synthesis encoded by transcripts generated by the E. coli RNAP (i.e. those made in the absence of rifampicin) decreased as the length of the template increased (FIG. 4a), as has been observed previously.

Additional experiments showed that as few as 5 base pairs upstream of the T7 promoter on the template DNA fragment were sufficient to promote efficient rifampicin-independent synthesis of CAT to a level that was much higher than that achieved by E. coli RNAP (FIG. 4c and 4d). This effect was independent of the composition of the nucleotide sequence of the 5′ base-pairs [the 20 base pair natural sequence vs. randomly inserted base pairs (20 N)] (FIG. 4c), and was due specifically to transcription by T7 RNAP (FIG. 4d).

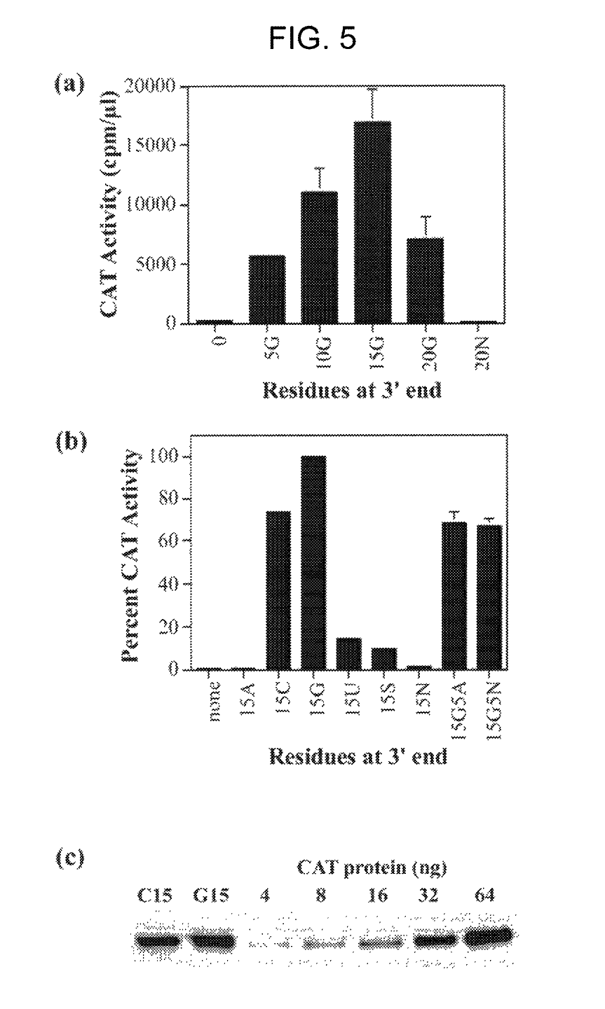

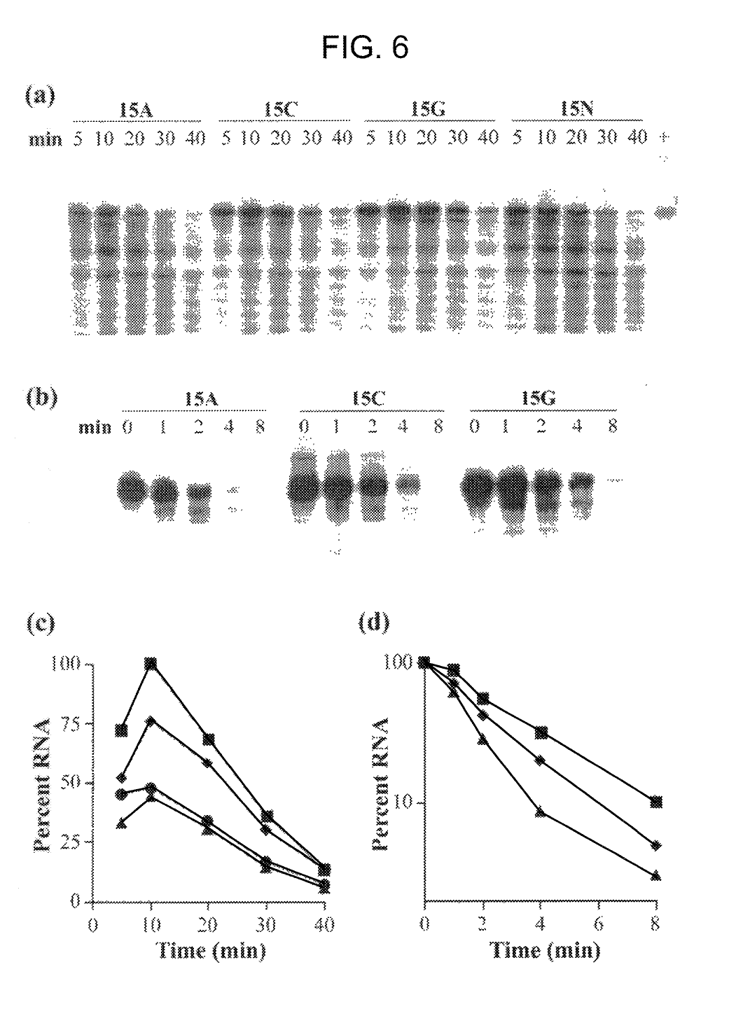

Stabilization of mRNA by a poly(G) tail during coupled transcription/translation in vitro: PCR-generated CAT gene templates containing the T7 promoter plus five additional upstream base pairs and 3′ sequences that generate different types of tails on the transcripts encoded by these templates, as indicated in FIGS. 5a and 5b, were tested for the ability to promote synthesis of active CAT protein in vitro during coupled transcription/translation. As seen, the number of G residues required for the maximum yield of translation product peaked at 15. Addition of the same length tail of homopolymeric C residues resulted in about 70% of the CAT protein production observed for CAT mRNA containing a poly(G) tail; interestingly, however, the effect of poly(C) tails was not observed for other mRNAs we tested and thus, does not appear to be general. The installation of 15 G residues internal to the transcript and 3′ to the ORF also resulted in enhancement of translation, yielding about 70% of protein production observed with 15 G residues at the 3′ terminus. The yield of CAT protein under the conditions of our assay was between 30 to 60 μg per ml of reaction, as determined by Western blot analysis (FIG. 5c). The observed steady state levels of mRNA (FIG. 6a) and the rate of RNA decay (FIG. 6b) for poly(G) and poly(C) tails correlated well with the effects of homopolymeric additions on CAT protein produced by transcription/translation of templates synthesized by PCR.

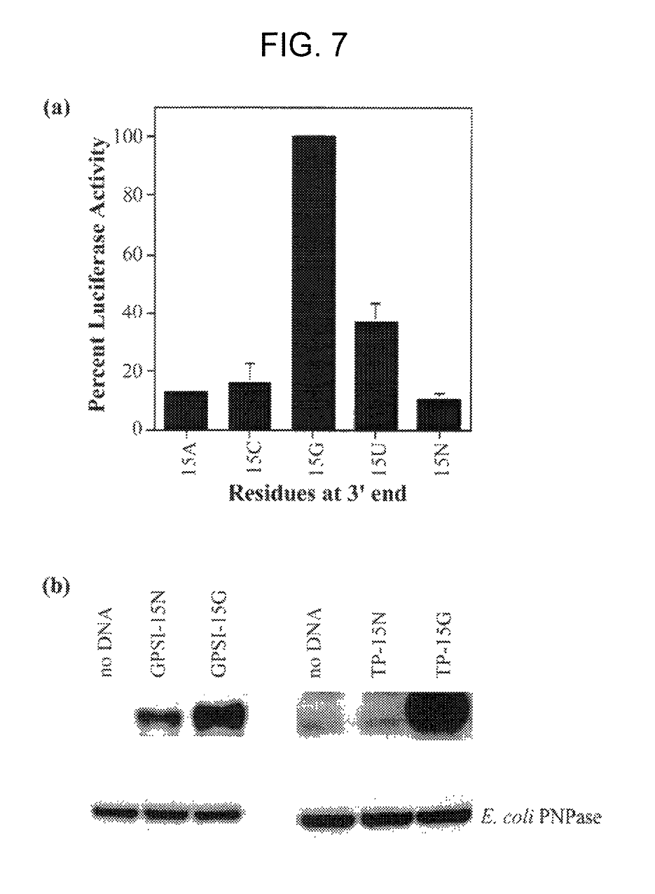

The ability of poly(G) tails installed on T7 polymerase-generated transcripts made in vitro in coupled transcription/translation reactions to stimulate production of the firefly luciferase protein was also tested and found to be similar to that observed for CAT (FIG. 7a). Additionally, the same approach has proved to be useful for the synthesis of proteins encoded by Streptomyces chromosomal ORFs (FIG. 7b), including those specifying guanosine pentaphosphate synthetase I (GPSI), an 80 kDa protein which is a homologue of E. coli PNPase and a 22 kDa S. rochei telomere binding protein (TP). Stimulation of production of 80 kDa GPSI by poly(G) tails was two-fold, while production of the 22 kDa TP was increased 22-fold by the poly(G) tail. For these proteins, as well as for luciferase and CAT, the extent of functional stabilization of transcripts by poly (G) was inversely related to transcript length.

Discussion

The data provided above revealed no evidence that 3′ polyadenylation alters the ability of transcripts to produce proteins in vitro. However, it was observed that 3′ poly (G) additions to transcripts can increase the chemical, and even more dramatically the functional half life of mRNA in E. coli cell extracts, yielding up to an 80-fold increase in the protein production.

Guanine-rich nucleic acids segments are known to form a structure called a “G quartet”, which commonly is found within telomeres. It previously was shown that G tails inhibit the binding and action of PNPase, one of two major 3′ to 5′ exonucleases of E. coli. However, poly(G) tails do not affect the cleavage by RNAse E, the principal endoribonuclease of E. coli, and in our experiments did not alter the rate or pattern of fragmentation of CAT mRNA in E. coli cell extracts. As the initial step in E. coli mRNA decay is believed to be endonucleotic cleavage by RNaseE, and the poly(G) tails do not affect the internal cleavage of transcripts by this enzyme, prolongation of the functional half-life of transcripts in vitro reaction mixtures by 3′ poly(G) additions was highly surprising. However, the ability of poly(G) tails to stabilize the protein-synthesizing ability of a variety of transcripts, together with our finding that poly(G) tails on CAT mRNA facilitate the synthesis of full length—rather then C-terminally truncated—CAT protein suggests that 3′ to 5′ decay—rather than endonucleolytic cleavage—was the rate limiting step that determined mRNA functional half life in the reaction mixtures we tested.

The effects of poly(G) tails on mRNA functional half life may not be explained entirely blockage of digestion by PNPase or other 3′ to 5′ exoribonucleases, as protein synthesis decreased when the length of the tail was extended past 15 nucleotides. Additionally, the protective effect of homopolymeric 3′ addition of G residues on transcript inactivation by 3′ to 5′ digestion may in part be masked when an mRNA has increased potential for internal cleavage by RNase E or other endonucleases, as the effect of poly (G) tail on the production of protein encoded by mRNA appeared to decrease as the length of the primary transcript increased.

We found during the course of our investigations that at least five additional non-specific base pairs 5′ to the bacteriophage T7 promoter are required for efficient transcription by the T7 RNAP. This effect and also the effect of poly(G) tails on mRNA functional half were observed also for a commercially-available transcription/translation reaction mixture (PROTEINscript-PRO™; Ambion) as well as for the cell extracts we prepared. Using as template a DNA that contained the CAT ORF and, (1) a 5′ primer that installed the T7 promoter and additional base pairs at the 5′ end of the PCR generated CAT ORF-containing template, and (2) a 3′ primer that installed a poly(G) tail on run-off transcripts synthesized by T7 polymerase, the reaction mixtures we prepared yielded a level of protein production that was comparable to protein levels reported for in vitro synthesis systems that use cloned genes on circular plasmid DNA as template. However, the ability to use polyguanylation to functionally stabilize transcripts made during in vitro transcription/translation, coupled with the ability to impart function to a T7 promoter sequence generated in templates by PCR amplification, obviates the use of cloned genes for in vitro protein synthesis, and may prove to be of considerable practical use during proteomic analysis of gene function.

What is claimed is:

1. A method for enhanced synthesis of a polypeptide in an expression system, the method comprising:
stabilizing mRNA encoding said polypeptide by insertion of a stabilizing sequence at the 3′ non-coding region of said mRNA,
translating said mRNA into its encoded protein in an in vitro expression system;
wherein said stabilized mRNA has an increased functional half-life and provides for enhanced protein synthesis.

2. The method of claim 1, wherein said stabilizing sequence comprises a poly (G) homopolymer of at least five nucleotides in length.

3. The method of claim 2, wherein said stabilizing sequence is inserted at the 3′ terminus of the mRNA.

4. The method of claim 3, wherein said stabilizing sequence is inserted by the method comprising:
amplifying a target sequence with primers, wherein a 3′ primer comprises a sequence that when transcribed provides a stabilization sequence.

5. The method of claim 4, wherein said primer is specific for a sequence of interest.

6. The method of claim 4, wherein said primer is a universal primer.

7. The method of claim 4, wherein said amplification is provided by polymerase chain reaction.

8. The method of claim 3, wherein said stabilizing sequence is inserted by the method comprising:
transcribing said mRNA from an expression vector wherein said stabilizing sequence is inserted 3′ region to the sequence encoding said polypeptide.

9. A method of enhancing transcription from a linear DNA with a phage specific promoter, the method comprising:
enhancing transcription by a phage specific RNA polymerase by insertion of a transcription enhancing sequences 5′ to said promoter.

10. The method of claim 9, wherein said transcription enhancing sequence comprises two or more nucleotides.

11. The method of claim 10, wherein said phage specific RNA polymerase is T7 RNA polymerase.

12. The method of claim 10, wherein said transcription enhancing sequence is inserted by the method comprising:
amplifying a target sequence with primers, wherein a 5′ primer comprises a transcription enhancing sequence.

13. The method of claim 12, wherein said primer is specific for a sequence of interest.

14. The method of claim 12, wherein said primer is a universal primer.

15. The method of claim 12, wherein said amplification is provided by polymerase chain reaction.

16. A method for synthesis of a targeted polypeptide, the method comprising:
amplifying a targeted polynucleotide sequence with a 5′ and a 3′ primer, wherein said 5′ primer comprises a T7 promoter and a transcription enhancing sequence 5′ to said promoter; and wherein said 3′ primer comprises an mRNA stabilization sequence; and wherein said primers specifically amplify said targeted polynucleotide sequence;
transcribing the amplification product into mRNA with T7 RNA polymerase;
translating said mRNA in vitro;
wherein said targeted polypeptide is synthesized.

17. The method according to claim 16, wherein said targeted polynucleotide sequence is present in a complex mixture of sequences.

18. The method of claim 17, wherein said complex mixture of sequences comprises multiple open reading frames.

19. The method of claim 16, further comprising analysis of said synthesized polypeptide by gel electrophoresis.

20. The method of claim 16, further comprising inserting an epitope tag in the coding sequence of said targeted polynucleotide sequence.

21. The method of claim 20, wherein said synthesized polypeptide is isolated by selective binding to said epitope tag.

22. A method for determining the identity of the protein product of a targeted polynucleotide, the method comprising:
amplifying a targeted polynucleotide sequence, wherein said targeted polynucleotide sequence is present in a complex mixture of sequences, with a 5′ and a 3′ primer, wherein said 5′ primer comprises a T7 promoter and a transcription enhancing sequence 5′ to said promoter; and wherein said 3′ primer comprises an mRNA stabilization sequence; and wherein said primers specifically amplify said targeted polynucleotide sequence;
transcribing the amplification product into mRNA with T7 RNA polymerase;
translating said mRNA in vitro; wherein said targeted polypeptide is synthesized;
combining said synthesized polypeptide with a cellular lysate;
analyzing said cellular lysate by gel electrophoresis; wherein the position of said synthesized identifies the protein product of the targeted polynucleotide.

23. A method for synthesizing polypeptides encoded by uncloned genomic open reading frames, the method comprising:
amplifying a targeted polynucleotide sequence, wherein said targeted polynucleotide sequence is an uncloned genomic open reading frame, with a 5′ and a 3′ primer, wherein said 5′ primer comprises a T7 promoter and a transcription enhancing sequence 5′ to said promoter; and wherein said 3′ primer comprises an mRNA stabilization sequence; and wherein said primers specifically amplify said targeted polynucleotide sequence; and wherein said primers insert an epitope tag into said genomic open reading frame;
transcribing the amplification product into mRNA with T7 RNA polymerase;
translating said mRNA in vitro; wherein said targeted polypeptide is synthesized;
isolating said synthesized polypeptide by binding to said epitope tag.


SEQUENCE LISTING (17 sequences)

SEQ ID NO: 1 - DNA, 27 bp, Artificial Sequence
  Primer
acaggatccatgaccatgattacggat

SEQ ID NO: 2 - DNA, 27 bp, Artificial Sequence
  Primer
acaggatccgtgcatctgccagtttga

SEQ ID NO: 3 - DNA, 42 bp, Artificial Sequence
  Primer
acaggatccaggaggctcgagatggagaaaaaaatcactgga

SEQ ID NO: 4 - DNA, 30 bp, Artificial Sequence
  Primer
acaggatccttacgccccgccctgccactc

SEQ ID NO: 5 - DNA, 19 bp, Artificial Sequence
  Primer
taatacgactcactatagg

SEQ ID NO: 6 - DNA, 22 bp, Artificial Sequence
  Primer
aaggctgttagcagccggatcc

SEQ ID NO: 7 - DNA, 19 bp, Artificial Sequence
  Primer
taatacgactcactatagg

SEQ ID NO: 8 - DNA, 20 bp, Artificial Sequence
  Primer
agatctcgatcccgcgaaat

SEQ ID NO: 9 - DNA, 18 bp, Artificial Sequence
  Primer
ttacgccccgccctgcca

SEQ ID NO: 10 - DNA, 60 bp, Artificial Sequence
  Primer
gaaattaatacgactcactatagggttaactttaagaaggagccaccatggaagacgcca

SEQ ID NO: 11 - DNA, 21 bp, Artificial Sequence
  Primer
ttacacggcgatctttccgcc

SEQ ID NO: 12 - DNA, 24 bp, Artificial Sequence
  Primer
gaaattaatacgactcactatagg

SEQ ID NO: 13 - DNA, 17 bp, Artificial Sequence
  Primer
tacgggacgtcactgct

SEQ ID NO: 14 - DNA, 73 bp, Artificial Sequence
  Primer
gaaattaatacgactcactatagggttaactttaagaaggagatatacatatggtggactcgatcggagacgg

SEQ ID NO: 15 - DNA, 45 bp, Artificial Sequence
  Primer
ctacttgtcgtcatcgtccttgtagtccagctggatctcgatctg

SEQ ID NO: 16 - DNA, 18 bp, Artificial Sequence
  Primer
ggatccgcgacccatttg

SEQ ID NO: 17 - DNA, 18 bp, Artificial Sequence
  Primer
gggttttcccagtcacga